(19) United States
(12) Patent Application Publication
(10) Pub. No.: US 2002/0111532 A1
(43) Pub. Date: Aug. 15, 2002

(54) Tris(hydroxymethyl)phosphino compounds as tissue crosslinking agents

(76) Inventors: Chandrashekhar P. Pathak, Austin, TX (US); Mark A. Moore, Austin, TX (US); Richard E. Phillips JR., San Marcos, TX (US); Alan Adams, Austin, TX (US)

Correspondence Address: Timothy Scott, Sulzer Medica USA Inc.,, 3 East Greenway Plaza, Suite 1600, Houston, TX, 77046-0391, US

(21) Appl. No.: 09/740,716

(22) Filed: Dec. 19, 2000

(51) Int. Cl. A61F 2/04

(52) U.S. Cl. 600/36

(57) ABSTRACT

A method of cross-linking a tissue, comprising treating the tissue under effective cross-linking conditions with a fluid comprising a compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorus atom. In one embodiment, the compound is β-(tris(hydroxymethyl)phosphino)proprionic acid (THPP).

Also disclosed is a cross-linked biological tissue produced by treating the tissue according to the above method.

### BACKGROUND OF THE INVENTION

1. Field of the Invention

The present invention relates generally to the field of preparing tissue for prosthetic use. More particularly, it concerns methods of crosslinking tissues with a tris(hydroxymethyl)phosphino compounds, such as β-(tris(hydroxymethyl)phosphino) proprionic acid.

2. Description of Related Art

Bioprostheses are devices derived from processed biological tissues to be used for implantation into a mammalian (e.g., human) host. Implantation of bioprostheses is a rapidly growing therapeutic field as a result of improvements in surgical procedures and immunosuppressive treatments, as well as increased knowledge of the graft-host interaction.

Several applications for tissue transplantation are known. For example, heart malfunction due to heart valve disorders can often be treated by surgically implanting a prosthetic heart valve. Treated tissue derived from porcine aortic valves or bovine pericardium is often used in the fabrication of such devices. Other tissue transplantation applications include tendons, ligaments, skin patches, pericardial patches, aortic patches, and tympanic membranes, among others. In the majority of known applications, the primary component of a bioprosthesis is collagen.

Several problems associated with tissue transplantation, include inflammation, degradation, calcification, and immune rejection. These problems are particularly common in transplantations involving collagen from a donor animal different from the transplantation recipient (i.e., non-autograft transplants). Attempts have been made to overcome these problems by tissue cross-linking (also referred to as “tissue fixation”) collagen molecules present in the implanted device. Cross-linking involves the use of bi- or multifunctional molecules having reactive groups capable of forming stable intra- and intermolecular bonds with reactive amino acid side groups present in the collagen of the bioprosthesis.

Glutaraldehyde is a bifunctional molecule capable of reacting under physiological conditions with the primary amine groups of collagen. Although it is the most commonly used chemical fixative for biological tissues, glutaraldehyde has a number of drawbacks associated with its use in cross-linking tissues for bioprosthetic use. For example, the long term durability of glutaraldehyde-fixed bioprostheses is poor, and there have been a number of reports of mechanical failures of glutaraldehyde-fixed tissue at points of high mechanical stress (Broom, 1977; Magilligan, 1988). Another drawback to glutaraldehyde fixation of bioprostheses is depolymerization of the cross-links in vivo, resulting in release of toxic glutaraldehyde into the host (Moczar et al., 1994; Wiebe et al., 1988; Gendler et al., 1984).

Further shortcomings of glutaraldehyde-cross-linking are related to the chemistry of the molecule. Glutaraldehyde forms a relatively unstable Schiff-base bond with collagen. In water, such as an aqueous solution of glutaraldehyde prior to performing a cross-linking treatment, glutaraldehyde can self-polymerize to form a water-soluble polyether polymer.

In addition, glutaraldehyde cross-linked bioprostheses have an undesirable propensity to calcify after implantation. This calcification is widely held to be the predominant cause of failure of glutaraldehyde-cross-linked devices (Golomb et al., 1987; Levy et al., 1986; Thubrikar et al., 1983; Girardot et al., 1995). Increased calcium uptake by a bioprosthesis typically leads to an accumulation of calcium phosphate, which in turn mineralizes into calcium hydroxyapatite. The calcification process is not well understood, but appears to depend on factors such as calcium metabolism diseases, age, diet, degeneration of tissue components such as collagen, and turbulence. Calcification of bioprostheses has been associated with degenerative changes in glutaraldehyde-treated collagen fibers.

A number of approaches have been investigated for reducing calcification of glutaraldehyde-fixed bioprostheses. For example, glutaraldehyde-fixed bioprosthetic heart valves have been treated with surfactants to reduce calcification after implantation (U.S. Pat. No. 5,215,541). In another approach, alpha-aminooleic acid treatment of glutaraldehyde-fixed tissue has been reported as an effective biocompatible, non-thrombogenic approach for minimizing calcification of bioprostheses (Girardot et al., 1991; Gott et al., 1992; Girardot et al., 1993; Hall et al., 1993; Myers et al., 1993; Girardot et al., 1994). The broad applicability of this approach in the production of bioprostheses, however, may be limited by the inability to achieve good tissue penetration by alpha-aminooleic acid into glutaraldehyde-fixed tissue (Girardot, 1994).

With respect to the biocompatibility of prosthetic devices, implantation of bioprostheses in living tissues typically initiates a series of physiological events which can activate host defense mechanisms such as coagulation, platelet adhesion and aggregation, white cell adhesion, and complement activation, among others. In attempts to improve the biocompatibility or hemocompatibility of articles adapted for use in contact with blood or blood products, aliphatic extensions have been added to the surface of bioprostheses in order to provide hydrophobic binding sites for albumin. The binding of albumin to a bioprosthesis has been reported to provide a low activation of coagulation, low complement activation, and reduced platelet and white cell adhesion, thereby providing improved hemocompatibility (U.S. Pat Nos. 5,098,960 and 5,263,992; Munro et al., 1981; Eberhart, 1989).

Some cross-linking agents have been investigated as alternatives to glutaraldehyde. These include polyepoxides, diisocyanates, di- and polycarboxylic acids, and photooxidation using organic dyes (see Khor, 1997, for review).

Therefore, a need exists within the field of bioprosthetics for simple, cost-effective methods for cross-linking biological tissues which provide bioprostheses with more desirable mechanical characteristics, reduced susceptibility to calcification, or enhanced biocompatibility relative to bioprostheses produced from glutaraldehyde-cross-linked tissue.

### SUMMARY OF THE INVENTION

In one embodiment, the present invention relates to a method of cross-linking a tissue, comprising treating the tissue under effective cross-linking conditions with a fluid comprising a tris(hydroxymethyl)phosphino compound.

In another embodiment, the present invention relates to a cross-linked biological tissue produced by treating the tissue under effective cross-linking conditions with a fluid comprising a tris(hydroxymethyl)phosphino compound.

The method allows cross-linking of tissues to an extent comparable to that seen for glutaraldehyde cross-linking. It has been surprisingly discovered that tissues crosslinked according to the method undergo greater endothelial cell growth than tissues crosslinked with glutaraldehyde. Accordingly, the present invention includes methods and compositions for improved biomaterials for use in implantable bioprostheses.

### DESCRIPTION OF THE FIGURES

FIG. 1 shows a 4-20% acrylamide:bisacrylamide (37.5:1 Mini-PROTEAN II) Gradient Ready Gel (Biorad, Richmond, Calif.) gel showing the results of protein extraction assays performed on bovine pericardium samples crosslinked with THPP, crosslinked with glutaraldehyde, or not crosslinked.

FIG. 2 shows a 4-20% acrylamide:bisacrylamide (37.5:1 Mini-PROTEAN II) Gradient Ready Gel (Biorad, Richmond, Calif.) gel showing the results of pepsin digestion assays (4 mg/mL pepsin (Sigma, St. Louis, Mo.) in 10 mM HCl for 4 hr at 37° C.) performed on bovine pericardium samples crosslinked with THPP, crosslinked with glutaraldehyde, or not crosslinked.

### DESCRIPTION OF ILLUSTRATIVE EMBODIMENTS

In one embodiment, the present invention relates to a method of cross-linking a tissue, comprising treating the tissue under effective cross-linking conditions with a fluid comprising a tris(hydroxymethyl)phosphino compound.

The tissue to be treated can be any tissue from which it is desired to fashion a bioprosthesis. A variety of tissues can be used, such as tendons, ligaments, heart valves, tissues usable to construct heart valves such as dura mater and pericardium, skin patches, pericardial patches, aortic patches, and tympanic membranes, among others. The tissue to be treated can be derived from any of a variety of animal species, such as humans, cattle, pigs, horses, sheep, rabbits, rats, ostriches, or kangaroos, among others.

By “tris(hydroxymethyl)phosphino compound” is meant any compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorous atom. The electrical charge on the P(CH₂OH)₃Xₙ, moiety can be 0, +1, or +2, and if charged, the moiety can be paired with a counterion to form a salt. An appropriate counterion is a halide, e.g. Cl⁻.

In one preferred embodiment, X is —(CH₂)₂COOH and n is 1, i.e., the tris(hydroxymethyl)phosphino compound is β-(tris(hydroxymethyl)phosphino) proprionic acid. In another preferred embodiment, n is 0, i.e. the tris(hydroxymethyl)phosphino compound is tris(hydroxymethyl)phosphine.

Without being bound by any particular theory, it is believed that the hydroxy groups of the compound undergo a Mannich-type condensation with amines present in the side chains of lysine or arginine residues found in collagen or other proteins present in the tissue. Other side reactions may occur.

Preferably, the tris(hydroxymethyl)phosphine compound is dissolved in a fluid solvent. The fluid comprising the compound also comprises a solvent. The solvent can be any liquid in which the compound is soluble and does not undergo degradation or side reactions. Preferably, the solvent is water or a buffered aqueous solution, or is a water-miscible organic solvent that has minimal toxicity to the tissue or the recipient, is non-denaturing, and is compatible with the cross-linking reaction. More preferably, the solvent is a buffered aqueous solution. A particularly preferred solvent is phosphate-buffered saline (PBS).

The concentration of the cross-linking agent in the fluid is preferably between about 0.1 mg/mL and about 100 mg/mL. More preferably, the concentration is between about 1 mg/mL and about 20 mg/mL.

The fluid can also comprise other additives that do not interfere with the cross-linking properties or other desirable properties of the fluid. Such additives include preservatives and adjuvants, among others.

The compound, as well as any other additives, can be synthesized by any known technique. For example, tris(hydroxymethyl)phosphine can be prepared by reacting PH₃ and formaldehyde in the presence of platinum salt catalysts, or by reacting P(CH₂OH)₄⁺Cl⁻ with triethyl amine (Ellis et al., 1992). Alternatively, the compound may be commercially available (e.g., THPP is commercially available from Pierce, Rockford, Ill.). The fluid can be prepared, typically, by dissolution of the compound, and any other additives, in the solvent. The fluid can be stored at any temperature and pH desired. The temperature and pH of storage need not be those which are effective for cross-linking. If necessary, prior to use, the pH and the temperature can be adjusted to the preferred ranges described below by known techniques.

The pH of the fluid can be any pH which is not deleterious to the tissue being treated or the cross-linking reaction. The pH of the fluid can be adjusted by any appropriate technique. Typically, the pH of the fluid is between about pH 6 and about pH 10. This pH range allows cross-linking to be relatively rapid and have a relatively low rate of side-reactions. Preferably, the pH of the fluid is between about pH 6.5 and about pH 8. More preferably, the pH of the fluid is between about pH 6.8 and about pH 7.5.

The temperature of the fluid can be any temperature at which the cross-linking reaction is relatively rapid and at which a relatively low rate of side reactions occur. Preferably, the temperature of the fluid is between about 0° C. and about 60° C. More preferably, the fluid temperature is between about 2° C. and about 30° C. Conveniently, the reaction may be carried out at room temperature (20-25° C.).

One of ordinary skill in the art will recognize that the duration of treatment is not critical, so long as the tissue and the cross-linking agent remain in contact long enough for cross-linking to proceed to a sufficiently great extent to obtain a cross-linked biomaterial suitable for use in implantable bioprostheses. The duration of treatment may vary depending on the tissue being treated or the particular tris(hydroxymethyl)phosphino compound being used for cross-linking. Typically, treatment duration is in the range of from about 1 min to about 24 hr. Preferably, treatment duration is at least about 30 min, more preferably at least about 6 hr.

The extent of cross-linking can be modified by varying any of several parameters, such as the tris(hydroxymethyl)phosphino compound used for cross-linking, pretreatment of the tissue with an agent that affects the cross-linking properties of the tris(hydroxymethyl)phosphino compound, the duration of treatment, the pH of treatment, the temperature of treatment, and other parameters known to persons skilled in the art of tissue cross-linking. The extent of cross-linking desired will depend on the physical properties and biocompatibility desired for a prosthesis made from the cross-linked tissue, among other properties apparent to persons of ordinary skill in the art.

The result of the cross-linking reaction is a cross-linked tissue suitable for use in a bioprosthesis. The cross-linked tissue can be formed into a bioprosthesis or a component of a bioprosthesis by methods known in the art. After the bioprosthesis is formed from the tissue, it can be implanted into an animal, preferably a mammal, according to known surgical procedures.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that changes, modifications, or additions can be made in the specific embodiments which are disclosed and still obtain a like or similar result without departing from the spirit and scope of the invention.

### EXAMPLE 1

### Cross Linking of Bovine Pericardium with β-(tris(hydroxymethyl)phosphino)proprionic Acid

Materials and Methods: β-(tris(hydroxymethyl)phosphino)proprionic acid (THPP) (Pierce, Rockford, Ill.) was dissolved in phosphate-buffered saline (PBS) at a concentration of 10 mg/mL. A control solution of 0.25% glutaraldehyde was prepared by diluting 10 mL of 8% electron microscope grade (EM) glutaraldehyde (Polysciences, Inc.) up to 320 mL with PBS.

Bovine pericardium was obtained from an abattoir and cleaned according to standard procedures in the art of bioprosthesis manufacturing. In particular, bovine pericardium tissue stored in a high salt/high sugar preservative solution (HSHS) as described in co-pending U.S. patent application Ser. No. 08/971,273, hereby incorporated by reference herein in its entirety, was obtained from the tissue manufacturing department of Sulzer Carbomedics.

Tissue Crosslinking: Bovine pericardium tissue was cut into ˜1 cm² samples and rinsed in ˜1 L ultrafiltered H₂O for about 30 min with stirring. The samples were then rinsed in PBS for an additional 30 min at room temperature with shaking.

After rinsing, samples were placed into either (a) the THPP solution given above; (b) the glutaraldehyde solution given above; or a control solution of (c) 0.01 M HEPES with 40% isopropyl alcohol. Samples were left in solution overnight at room temperature. Samples were then rinsed 5×30 min in ˜200 mL PBS each rinse, and then either tested immediately or stored in 0.01 M HEPES with 40% isopropyl alcohol until testing.

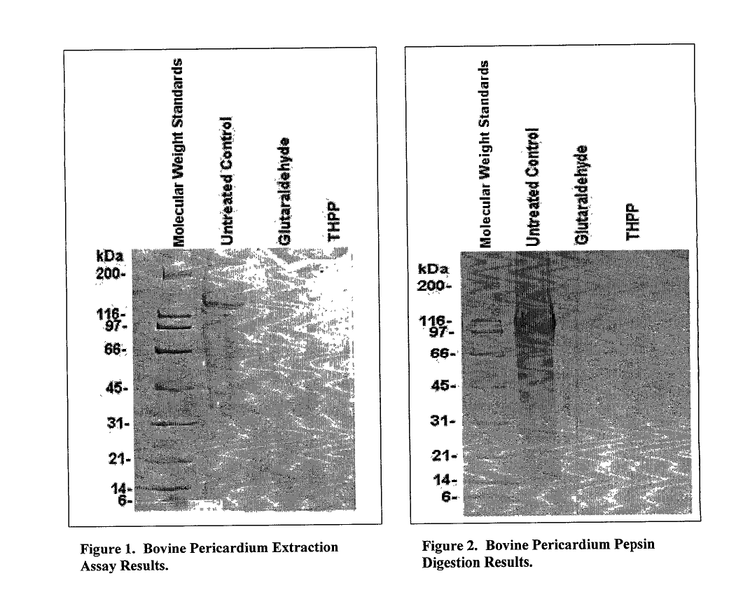

Protein Extraction Assay: Crosslinked samples of the THPP treated, glutaraldehyde treated, and untreated control tissues underwent protein extraction assays using a standard procedure established at Sulzer Carbomedics. To summarize, 10-20 mg of each of the three tissue sample types (THPP treated, glutaraldehyde treated, untreated control) was extracted with a 10-20 μL extraction solution containing 50 mM Tris-HCl (pH 6.8), 10% glycerol, 4% mercaptoethanol, 1% sodium dodecyl sulfate (SDS), 0.5 M NaCl, and 0.01% bromophenol blue. The extracted solution was then run on a 4-20% acrylamide:bisacrylamide (37.5:1 Mini-PROTEAN II) Gradient Ready Gel (Biorad, Richmond, Calif.). The extract gel, including reference standards, is shown in FIG. 1. The gel also underwent densitometry scanning. QuantiScan for Windows (densitometer program) was used to calculate peak areas of the protein bands on the gel. The areas were used to calculate the fraction of each extracted protein relative to all proteins present.

Pepsin Digestion Assay: Some crosslinked samples were digested in 4 mg/mL pepsin (Sigma, St. Louis, Mo.) in 10 mM HCl for 4 hr at 37° C. Enzyme:tissue ratios (weight:wet weight) were 1:2500. Following centrifugation at room temperature for ˜5 min at 13,000 rpm (30,000 g), reaction supernatants were retained for gel electrophoresis. Polyacrylamide gel electrophoresis was performed on a 4-20% acrylamide:bisacrylamide (37.5:1 Mini-PROTEAN II) Gradient Ready Gel (Biorad, Richmond, Calif.). The digestion gel, including reference standards, is shown in FIG. 2.

Shrinkage Temperature Assay: The shrinkage temperature of some crosslinked samples was determined using standard differential scanning calorimetric analysis. Briefly, 2-10 mg of tissue was heated at 10° C. increments under nitrogen. Typically, an endotherm is seen in the range of 60° C.-90° C., and this endotherm is attributed to a shrinkage transition.

Attachment and Spreading of Bovine Endothelial Cells on Crosslinked Bovine Pericardial Tissue: THPP-fixed bovine pericardial tissue and glutaraldehyde-fixed bovine pericardial tissue (control) were used in this experiment. Tissue samples (1 cm×1 cm) were soaked overnight in 70% ethanol, washed 3×with sterile PBS (15 min per wash), and soaked in Minimum Essential Medium (MEM), supplemented with amino acids, antibiotics, and 30% fetal bovine serum (FBS) as is well known in the art of growing endothelial cells, for 1.5 hr. (MEM is available from Gibco BRL, Life Technologies, catalog number 11095-080). The tissue samples were then transferred to a 24 well sterile tissue culture plate. Bovine aortic endothelial cells were passaged in MEM/10% FBS, resuspended in MEM/10% FBS, and approximately 200,000 cells were placed on the tissue samples. The cells were allowed to adhere for 30 min, and then 0.75 mL of the same medium was added to each sample.

The samples were then incubated 24 hr in an atmosphere of 5% carbon dioxide/95% air at 98% humidity and 37° C. The samples were then transferred to new wells and fresh medium was added to the new wells. The samples were incubated for a further 24 hr. At the end of this incubation period, the tissue samples and cells were washed with PBS (3×, 5 min per wash, room temperature), and fixed using 4% paraformaldehyde for 10 min. The samples were then rinsed in PBS (3×, 5 min per wash, room temperature) and treated with 0.1% Triton X100 in PBS for 3 min. The cells were then stained with phalloidin/rhodamine (diluted 1:40 in PBS) in the dark for 45 min, rinsed 3× in PBS, and viewed immediately under a fluorescence microscope. The morphology and coverage of endothelial cells on tissue samples were recorded, and representative photographs taken.

Results: In the protein extraction assay gel shown in FIG. 1, the untreated control showed far more extracted proteins than either THPP- or glutaraldehyde-treated tissue. THPP-treated tissue showed less extracted protein compared to untreated tissue, and an amount comparable to glutaraldehyde-treated tissue. The low levels of extracted protein in the treated tissues indicate a high degree of crosslinking. Densitometry scan data also indicated a relatively low level of extracted proteins in the THPP- and glutaraldehyde-treated tissues.

In the pepsin digestion assay gel, THPP-treated tissue and glutaraldehyde-treated tissue showed comparable and significant reductions in solubilized protein after pepsin digestion compared to the control, as shown in FIG. 2. The reductions in solubilized protein indicate stabilization of the tissue against enzymatic digestion in both THPP- and glutaraldehyde-treated tissue.

The protein extraction assay and the shrink temperature assay results are quantified in the following table.

TABLE 1
Extraction Assay and Shrink Temperature Assay Results

| | Extracted Protein | Shrink Onset |
|---|---|---|
| Treatment | (negative control = 100%) | Temperature (° C.) |
| None (unfixed tissue) | 100.00 | 66.94 |
| Glutaraldehyde | 1.74 | 86.04 |
| THPP | 1.59 | 77.93 |

In summary, THPP was about as effective in cross-linking (as determined from extracted protein and shrink onset temperature) as glutaraldehyde.

Also, THPP-fixed tissue supported the growth of bovine endothelial cells to a greater extent than did glutaraldehyde-fixed tissue. The number of endothelial cells attached to each fixed tissue surface is shown in Table 2.

TABLE 2
Attachment and spreading of bovine endothelial cells to fixed surfaces

| | Number of endothelial cells per field |
|---|---|
| | (high magnification) attached to tissue |
| Tissue Type | surface (n = 5) |
| glutaraldehyde-fixed tissue | 2.0 ± 1.1 |
| THPP-fixed tissue | 4.8 ± 0.4 |

The glutaraldehyde-fixed tissue had fewer cells attached thereto as compared with THPP-fixed tissue (statistically significant, t test). It is known that glutaraldehyde-fixed tissue surfaces cannot support endothelial cells, possibly due to leaching of cytotoxic glutaraldehyde from the tissue.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention, as defined by the appended claims.

### References

Broom, J. Biomech. 10:707 (1977)

Eberhart, IEEE Eng. in Med. and Bio. 26 (June 1989)

Ellis et al., Inorg. Chem., 31:3026 (1992)

Frautschi, U.S. Pat. No. 5,098,960

Gendler et al., J. Biomed. Mater. Res. 18:727 (1984)

Girardot et al., Trans. Soc. Biomat. 14:114 (1991)

Girardot et al., Trans. Soc. Biomat. 16:266 (1993)

Girardot et al., Int. J. Artif. Org. 17:76 (1994)

Girardot et al., J. Biomed. Mater. Res. 29:793 (1995)

Girardot, J. M., et al. J. Heart Valve Dis. 5:518 (1996)

Golomb et al., Am. J. Pathol 127:122 (1987)

Gott et al., Ann. Thorac. Surg. 53:207 (1992)

Guire, U.S. Pat. No. 5,263,992

Hall et al., ASAIO Proc. 24 (1993)

Khor, E., Biomaterials 18:95 (1997)

Levy et al., in: Williams, D. F., Ed. CRC Critical Rev. in Biocompatibility, Vol. 2. 147 (1986)

Magilligan, Trans. Am. Soc. Artif Intern. Organs 34:1031 (1988)

Moczar et al., ASAIO J. 40:M697 (1994)

Moore, M. A., et al. Ann. Thorac. Surg. 66:S245 (1998)

Munro et al., Trans. Am. Soc. Artif. Intern. Organs 27:499 (1981)

Myers et al., Int. J. Artif. Org. 16:453 (1993)

Nashef et al., U.S. Pat. No. 5,215,541

Petach, H. H., et al. J. Chem. Soc., Chem. Commun. 2181 (1994)

Sung, H. W., J. Biomed. Mater. Res. 42:560 (1998)

Thubrikar et al., J. Thorac. Cardiovasc. Surg. 86:115 (1983)

Wiebe et al., Surgery 104:26 (1988)

Zeeman, R., et al. J. Biomed. Mater. Res. 46:424 (1999)

What is claimed is:

1. A method of cross-linking a tissue, comprising:
A) providing a tissue to be cross-linked;
B) providing a cross-linking fluid comprising a compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorous atom; and
C) contacting the tissue with the cross-linking fluid under conditions effective to cross-link said tissue.

2. The method of claim 1, wherein X is —(CH₂)₂COOH and n is 1.

3. The method of claim 1, wherein n is 0.

4. The method of claim 1, wherein the tissue is derived from an animal of species humans, cattle, pigs, horses, sheep, rats, rabbits, ostriches, or kangaroos.

5. The method of claim 1, wherein the tissue comprises tendon, ligament, heart valve, dura mater, pericardium, skin patch, pericardial patch, aortic patch, or tympanic membrane tissue.

6. The method of claim 1, wherein the fluid further comprises a solvent.

7. The method of claim 6, wherein the solvent is phosphate-buffered saline (PBS).

8. The method of claim 1, wherein the fluid has a pH between about pH 6 and about pH 10.

9. The method of claim 8, wherein the fluid has a pH between about pH 6.5 and about pH 8.

10. The method of claim 9, wherein the fluid has a pH between about pH 6.8 and about pH 7.5.

11. The method of claim 1, wherein the fluid has a temperature between about 0° C. and about 60° C.

12. The method of claim 11, wherein the fluid has a temperature between about 2° C. and about 30° C.

13. The method of claim 1, wherein the treating is performed for at least about 30 min.

14. The method of claim 13, wherein the treating is performed for at least about 6 hr.

15. A method of forming a bioprosthesis of a cross-linked tissue, comprising:
A) providing a tissue to be cross-linked;
B) providing a cross-linking fluid comprising a compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorous atom;
C) contacting the tissue with the cross-linking fluid under conditions effective to cross-link said tissue to obtain a cross-linked tissue; and
D) incorporating the cross-linked tissue into a bioprosthesis.

16. The method of claim 15, further comprising implanting the bioprosthesis into an animal.

17. A cross-linked biological tissue produced by treating the tissue under effective cross-linking conditions with a fluid comprising a compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorous atom.

18. The cross-linked tissue of claim 17, wherein X is —(CH₂)₂COOH and n is 1.

19. The cross-linked tissue of claim 17, wherein n is 0.

20. The cross-linked tissue of claim 17, wherein the tissue is derived from an animal of species humans, cattle, pigs, horses, sheep, rats, rabbits, ostriches, or kangaroos.

21. The cross-linked tissue of claim 17, wherein the tissue comprises tendon, ligament, heart valve, dura mater, pericardium, skin patch, pericardial patch, aortic patch, or tympanic membrane.

22. The cross-linked tissue of claim 17, wherein the fluid further comprises a solvent.

23. The cross-linked tissue of claim 22, wherein the solvent is phosphate-buffered saline (PBS).

24. The cross-linked tissue of claim 17, wherein the fluid has a pH between about pH 6 and about pH 10.

25. The cross-linked tissue of claim 24, wherein the fluid has a pH between about pH 6.5 and about pH 8.

26. The cross-linked tissue of claim 25, wherein the fluid has a pH between about pH 6.8 and about pH 7.5.

27. The cross-linked tissue of claim 17, wherein the fluid has a temperature between about 0° C. and about 60° C.

28. The cross-linked tissue of claim 27, wherein the fluid has a temperature between about 2° C. and about 30° C.

29. The cross-linked tissue of claim 17, wherein the treating is performed for at least about 30 min.

30. The cross-linked tissue of claim 29, wherein the treating is performed for at least about 6 hr.

31. A bioprosthesis comprising a cross-linked biological tissue produced by treating the tissue under effective cross-linking conditions with a fluid comprising a compound comprising P(CH₂OH)₃Xₙ, wherein X is selected from C₁-C₁₀ carboxyl, sulfonic acid, sulfonic acid salts, C₁-C₁₀ alcohol, or halogens, and n is an integer from 0 to 2, inclusive, and all —X and —CH₂OH groups are bonded to the phosphorus atom.
